(19) United States
(12) Patent Application Publication
(10) Pub. No.: US 2024/0091175 A1
(43) Pub. Date: Mar. 21, 2024

(54) METHODS OF CONTROLLING MYOCARDIAL BLOOD FLOW

(71) Applicant: UNIVERSITY OF LOUISVILLE RESEARCH FOUNDATION, INC., Louisville, KY (US)

(72) Inventors: Matthew Nystoriak, Floyds Knobs, IN (US); Aruni Bhatnagar, Prospect, KY (US)

(73) Assignee: UNIVERSITY OF LOUISVILLE RESEARCH FOUNDATION, INC., Louisville, KY (US)

(21) Appl. No.: 18/370,633

(22) Filed: Sep. 20, 2023

Related U.S. Application Data
(63) Continuation of application No. 17/577,821, filed on Jan. 18, 2022, now abandoned.
(60) Provisional application No. 63/138,308, filed on Jan. 15, 2021.

(51) Int. Cl. A61K 31/137; A61P 9/08

(52) U.S. Cl. CPC A61K 31/137; A61P 9/08

(57) ABSTRACT

In certain embodiments, the present invention provides a method of modulating myocardial blood flow (MBF) as compared to a control in a patient in need thereof, comprising administering an agent that interacts with a Kvβ protein.

### CROSS-REFERENCE TO RELATED APPLICATIONS

This application is a continuation of U.S. patent application Ser. No. 17/577,821, filed Jan. 18, 2022, and claims priority to U.S. Provisional Application No. 63/138,308, filed Jan. 15, 2021. The entire content of the applications referenced above are hereby incorporated by reference herein.

### STATEMENT REGARDING FEDERALLY SPONSORED RESEARCH

This invention was made with government support under HL 142710 and GM103492 awarded by National Institutes of Health. The government has certain rights in the invention.

### BACKGROUND

An imbalance between myocardial oxygen supply and demand is a salient feature of heart disease, which remains the leading cause of death worldwide. Impaired cardiac function associated with inadequate myocardial perfusion is commonly observed in patients with heart failure, hypertension, diabetes, and coronary artery disease. Even in the absence of stenoses in large diameter conduit arteries, suppressed vasodilator capacity of small diameter coronary arteries and arterioles can lead to ischemia. Despite the vital importance of oxygen delivery to the preservation of cardiac structure and function, the fundamental mechanisms by which the coronary vasculature responds to fluctuations in myocardial metabolic demand remain poorly understood.

In the healthy heart, the coronary arteries and arterioles remain partially constricted, and they dilate or constrict further according to myocardial requirements for oxygen and nutrient delivery. As myocardial oxygen consumption increases (e.g., due to an increase in heart rate, myocardial contractility, or afterload), there is a corresponding demand for an increase in oxygen supply to sustain oxidative energy production. However, with little reserve for increased oxygen extraction, sustained cardiac function relies on the intimate link between local and regional metabolic activity and vasodilation of the coronary vascular bed to deliver adequate blood flow to the myocardium (i.e., metabolic hyperemia). In searching for molecular entities that couple vascular function to myocardial oxygen demand, recent studies have found that increased cardiac work promotes coronary vasodilation and hyperemia via the activation of Kv1 channels in smooth muscle cells. Nonetheless, how vascular Kv1 channels sense changes in oxygen demand to regulate blood flow to the heart is unclear.

### SUMMARY

In one aspect, provided herein is a method of modulating myocardial blood flow (MBF) as compared to a control in a patient in need thereof, comprising administering an agent that interacts with a Kvβ protein.

In one aspect, the Kvβ protein is a Kvβ1 protein.

In one aspect, Kvβ protein is a Kvβ2 protein.

In one aspect, provided herein is a method of suppressing myocardial blood flow (MBF) as compared to a control in a patient in need thereof, comprising administering an agent that inhibits the Kvβ2 protein.

In one aspect, provided herein is a method of suppressing myocardial blood flow (MBF) as compared to a control in a patient in need thereof, comprising administering an agent that inhibits the Kvβ1 protein.

In one aspect, provided herein is a method of impairing cardiac contractile performance as compared to a control in a patient in need thereof, comprising administering an agent that inhibits the Kvβ2 protein

In one aspect, provided herein is a method of impairing arterial blood pressure as compared to a control in a patient in need thereof, comprising administering an agent that inhibits the Kvβ2 protein.

In one aspect, provided herein is a method of reducing cardiac workload as compared to a control in a patient in need thereof, comprising administering an agent that inhibits the Kvβ1 protein.

In one aspect, provided herein is a method of preserving cardiac function during stress as compared to a control in a patient in need thereof, comprising administering an agent that inhibits the Kvβ1 protein.

In one aspect, provided herein is a method of inducing enhancement of Kvβ1:Kvβ2 ratio in Kv1 channels of arterial smooth muscle abolished L-lactate-induced vasodilation and suppressed the relationship between MBF and cardiac workload as compared to a control comprising administering an agent that interacts with a Kvβ protein.

### BRIEF DESCRIPTION OF DRAWINGS

The patent or application file contains at least one drawing executed in color. Copies of this patent or patent application publication with color drawings will be provided by the Office upon request and payment of the necessary fee.

FIGS. 1A-1C: Loss of Kvβ2 impairs cardiac pump function during stress. (A) Representative M-mode echocardiographic images obtained from wild type (WT; 129SvEv), and Kvβ2^{-/-} mice during infusion of 5 μg/kg·min⁻¹ norepinephrine. (B) Box-and-whisker plot of ejection fraction data for WT and Kvβ2^{-/-} mice at baseline, after administration of hexamethonium (HX; 5 mg·kg⁻¹, i.v.), and during norepinephrine infusions (0.5-5 μg/kg·min⁻¹; 2-3 min duration). n=8 each, **P<0.01, ***P<0.001 (two-way RM ANOVA). (C) Arterial blood pressure recordings obtained via femoral artery catheter in WT and Kvβ2^{-/-} mice, before and after norepinephrine treatment (NE, 5 μg/kg·min⁻¹, indicated by arrows).

FIGS. 2A-2D: Relationship between myocardial blood flow and cardiac workload in Kvβ-null mice. (A) Long axis MCE images showing signal intensity from myocardial tissue and cavity before destruction frame and during replenishment phase (˜10 sec). The left ventricular wall is outlined with a yellow dashed line in the destruction frame. (B) Signal intensity versus time (following destruction frame) in region of interest in the anterior left ventricular myocardial wall of WT (129SvEv), Kvβ1.1^{-/-}, and Kvβ2^{-/-} mice. Data were fit with exponential function (see inset). (C,D) Summary of MBF as a function of cardiac workload (double product; heart rate×mean arterial pressure) in Kvβ1.1^{-/-} (C) and Kvβ2^{-/-} (D) versus strain-matched wild type (WT) control mice. Data were fit with a simple linear regression model with slopes: WT (0.00192±0.00031), Kvβ1.1^{-/-} (0.00279±0.00016); n=6-8 mice; WT (0.00241±0.00014), Kvβ2^{-/-} (0.00162±0.00022); n=4-8 mice, *P<0.05, slope of Kvβ2^{-/-} vs. WT.

FIGS. 3A-3D: Ablation of Kvβ2 attenuates hypoxia-induced coronary vasodilation. (A) Summarized bath O₂(%) measured in normoxic and hypoxic conditions (perfusate aerated with 5% CO₂, balance N₂, +1 mM Na₂S₂O₄); data are pooled from measurements obtained with wild type (129SvEv) and Kvβ2^{-/-} coronary arteries. n=7-9, ***P<0.001 (Mann Whitney U). (B) Representative arterial diameter recordings in isolated preconstricted (100 nM U46619) coronary arteries from wild type (WT; 129SvEv) and Kvβ2^{-/-} mice in normoxic and hypoxic conditions. Ca²⁺-free perfusate containing nifedipine (nifed; 1 μM) and forskolin (fsk; 0.5 μM) was introduced at the end of the experiment to induce maximum dilation. (C) Scatter-plot and mean±SEM showing percent decrease in diameter recorded under normoxic (− hypoxia) and hypoxic (+ hypoxia) conditions for arteries from WT and Kvβ2^{-/-} mice. Normoxic and hypoxic conditions were both applied in continuous presence of U46619, see above (B). n=5 arteries, 3-4 mice *P<0.05, ns: P≥0.05 (one-way ANOVA, Tukey). (D) Scatter-plot and mean±SEM showing hypoxia-induced dilation (%) for arteries from WT and Kvβ2^{-/-} mice. **P<0.01 (Mann-Whitney U test).

FIGS. 4A-4I: L-lactate enhances hi, in coronary arterial myocytes and promotes coronary vasodilation via Kvβ2. (A, B) Representative outward K⁺ current recordings normalized to cell capacitance (pA/pF) in response to step-wise (10 mV) depolarization to +50 mV from a holding potential of −70 mV in isolated coronary arterial myocytes. Currents were recorded before and after application of 10 mM L-lactate in bath solution lacking (A) or containing (B) 500 nM psora-4. (C, D) Summary current-voltage relationships obtained in coronary arterial myocytes before and after application of 10 mM L-lactate in bath solution lacking (C) or containing (D) 500 nM psora-4. n=5-7 cells from 4-7 mice. *P<0.05, ns: P≥0.05 (two-way RM ANOVA). (E) Summary of L-lactate-induced currents recorded in the absence and presence of 500 nM psora-4. n=5-7 cells from 4-7 mice. *P<0.05 (mixed-effects). (F-H) Arterial diameter traces obtained from pressurized (80 mmHg) coronary arteries isolated from wild type (WT; 129SvEv; F,G) and Kvβ2^{-/-} (H) mice in the absence and presence of L-lactate (5-20 mM, as indicated). Arteries were preconstricted with 100 nM U46619; for WT arteries, L-lactate was applied in the absence (top) and presence (bottom) of psora-4 (500 nM). Maximum passive diameter was recorded at the end of each experiment in Ca²⁺-free saline solution with nifedipine (nifed; 1 μM) and forskolin (fsk; 0.5 μM). (I) Summary plot showing L-lactate-induced dilation, expressed as a percent change from baseline diameter relative to maximum passive diameter, for arteries isolated from WT (129SvEv; ±500 nM psora-4) and Kvβ2^{-/-} mice. n=4 arteries from 4 mice for each. *P<0.001; ns: P≥0.05, lactate vs. baseline (Friedman).

FIGS. 5A-5D: Kvβ2 controls redox-dependent vasoreactivity in resistance mesenteric arteries. (A) Representative fluorescence images showing PLA-associated fluorescent punctae (red) in wild type coronary and mesenteric arterial myocytes. Cells were labelled for Kv1.5 alone, or co-labelled for Kv1.5 and Kv1.2, Kv1.5 and Kvβ1.1, Kv1.5 and Kvβ2, or Kvβ1.1 and Kvβ2 proteins. DAPI nuclear stain is shown for each condition (blue). Scale bars represent 5 μm. (B) Summary of PLA-associated punctate sites normalized to total cell footprint area for conditions and groups as in D. P values are shown for coronary versus mesenteric arteries (Mann Whitney U). (C,D) Arterial diameter traces obtained from pressurized (80 mmHg) mesenteric arteries isolated from wild type (C; 129SvEv) and Kvβ2^{-/-} (D) mice in the absence and presence of L-lactate (5-20 mM, as indicated). Arteries were preconstricted with 100 nM U46619 and L-lactate was applied in the absence (top) and presence (bottom) of the selective Kv1 channel inhibitor psora-4 (500 nM). Maximum passive diameters were recorded at the end of each experiment in Ca²⁺-free saline solution with nifedipine (nifed; 1 μM) and forskolin (fsk; 0.5 μM). (E) Summary plot of L-lactate-induced dilation, expressed as the percent change from baseline diameter relative to maximum passive diameter, for arteries isolated from WT (129SvEv; ±psora-4) and Kvβ2^{-/-} mice. n=5 arteries from 4-5 mice for each. *P<0.05; ns: P≥0.05, lactate vs. baseline (Friedman).

FIG. 6A-6H: Increasing the ratio of Kvβ1.1:Kvβ2 subunits in smooth muscle inhibits L-lactate-induced vasodilation and suppresses myocardial blood flow. (A) Schematic diagram describing the SM22α-rtTA:TRE-β1 model. Double transgenic animals (+dox) results in activation of the reverse tetracycline trans-activator (rtTA) in smooth muscle cells, and drives expression of Kvβ1.1. (B) Western blots showing immunoreactive bands for Kvβ1 in whole mesenteric artery and brain lysates from SM22α-rtTA (single transgenic control) and SM22α-rtTA:TRE-β1 (double transgenic) mice after doxycycline treatment. Ponceau-stained membrane (mol. Wt.: ˜30-55 kDa) is shown as an internal control for total loaded protein. (C) Summarized relative densities of Kvβ1.1-associated immunoreactive bands in mesenteric arteries and brains of SM22α-rtTA:TRE-β1 relative to SM22α-rtTA. n=3 each. *P<0.05, ns: P≥0.05 (one sample t test). (D) Representative fluorescence images showing PLA-associated fluorescent punctae (red) in coronary arterial myocytes isolated from SM22α-rtTA and SM22α-rtTA:TRE-β1 mice. Cells were labelled for Kv1.5 alone, or co-labelled for Kv1.5 and Kvβ1, or Kv1.5 and Kvβ2 proteins. DAPI nuclear stain is shown for each condition (blue). Scale bars represent 5 μm. (E) Summary of PLA-associated punctate sites normalized to total cell footprint area for conditions and groups as in D. n=6-19 cells from 2-3 mice for each; *P<0.05, **P<0.001 (Mann Whitney U). (F) Representative arterial diameter recordings from 100 nM U46619-preconstricted mesenteric arteries isolated from SM22α-rtTA and SM22α-rtTA:TRE-β1 mice in the absence and presence of L-lactate (5-20 mM), as in FIG. 5C,D. Passive dilation in the presence of Ca²⁺-free solution+nifedipine (1 μM) and forskolin (fsk; 0.5 μM) is shown for each recording. (G) Summary plot of L-lactate-induced dilation for arteries isolated from SM22α-rtTA and SM22α-rtTA:TRE-β1 mice. n=6-10 arteries from 5-6 mice; *P<0.05; ns: P≥0.05, lactate vs. baseline (Friedman). (H) Summary relationships between myocardial blood flow (MBF) and cardiac workload (double product; heart rate×mean arterial pressure) in SM22α-rtTA:TRE-β1 vs. SM22α-rtTA control mice. n=5 each; ***P<0.001 (linear regression).

FIGS. 7A-7B. Heart rate and mean arterial pressure in wild type and KIT-null mice during catecholamine-induced stress. (A, B) Summary graphs showing heart rate (HR; left) and mean arterial pressure (MAP; right) in Kvβ1.1^{-/-} (A) and Kvβ2^{-/-} (B) and strain-matched wild type (WT) mice at baseline (0 μg/kg·min⁻¹ NE) and during intravenous infusion of norepinephrine (0.5-5 μg/kg·min⁻¹). WT, Kvβ1.1^{-/-}: n=6-8 mice; WT, Kvβ2^{-/-}: n=4-8 mice *P<0.05, ns: P≥0.05 (two-way RM ANOVA).

FIGS. 8A-8B. Hypoxia-induced vasodilation of isolated coronary arteries is attenuated in the presence of psora-4. (A) Representative coronary arterial diameter recordings obtained in the absence and presence of either 1 mM sodium hydrosulfite aerated with 95% N₂/0% O₂ (0% O₂+Na₂S₂O₄; i.), 1 mM sodium hydrosulfite aerated with 20% O₂ (20% O₂+Na₂S₂O₄; ii.), or 1 mM sodium hydrosulfite aerated with 95% N₂/0% O₂ applied in the presence of 500 nM psora-4 (iii.). (B) Summary of normalized % change in arterial diameter for conditions as indicated in A. n=4-5 arteries from 4-5 mice, *P<0.05, ***P<0.001, (one-way ANOVA with Dunnett's post-hoc test).

FIGS. 9A-9G. Loss of Kvβ subunits does not impact vasoconstriction in response to increases in intravascular pressure, membrane depolarization, or thromboxane A₂ receptor activation. (A,B) Summarized % decrease in diameter at intravascular pressures of 20, 40, 60, 80, and 100 mmHg for mesenteric arteries from WT (C57Bl6N) and Kvβ1.1^{-/-} mice (A; n=4-5 arteries from 4-5 mice), and WT (129SvEv) and Kvβ2^{-/-} mice (B; n=5 arteries from 4 mice each); ns: P≥0.05 (two-way RM ANOVA). (C) Symbol plots showing summarized passive diameters, obtained in Ca²⁺-free bath solution containing 0.5 μM forskolin and 1 μM nifedipine, relative to diameters at 0 mmHg across the range of intravascular pressures tested for arteries from Kvβ1.1^{-/-}, Kvβ2^{-/-}, and corresponding WT mice. n=4-5 arteries from 4-5 mice each. ns: P≥0.05 (two-way RM ANOVA). (D-G) Scatter plots summarizing % decrease in diameter obtained from mesenteric arteries before and after application of 60 mM [K⁺]ₒ (D, F) and 100 nM U46619 (E, G) in Kvβ1.1^{-/-}, Kvβ2^{-/-}, and corresponding WT mice. [K⁺]ₒ, (D) n=4-5 arteries from 4-5 mice, (F) n=5-6 arteries from 4-5 mice; U46619, (E) n=11 arteries from 10-11 mice, (G) n=10 arteries from 9-10 mice. ns: P≥0.05 (Mann Whitney U).

FIGS. 10A-10D. Vasodilation in response to L-lactate is independent of endothelial function and requires changes in membrane potential. (A) Arterial diameter recordings from preconstricted (100 nM U46619) intact and endothelium-denuded (-endo) arteries in the absence and presence of the SK_{Ca}/IK_{Ca} opener NS309 (1 μM; top) and absence and presence of 10 mM L-lactate (bottom). (B) Summarized percent change in diameter in response to 10 mM L-lactate in intact and-endo arteries. n=4−arteries from 3-4 mice; n>0.05 (Mann-Whitney U). (C) Arterial diameter traces from pressurized (80 mmHg) mesenteric arteries isolated from wild type mice preconstricted with either U46619 (100 nM) or high [K⁺]ₒ (60 mM), before and after application of 10 mM L-lactate. (D) Summarized 10 mM L-lactate-induced vasodilation (percent of maximal dilation) in arteries preconstricted with either 100 nM U46619 or with high [K⁺]ₒ. n=4-6 arteries from 4-5 mice; *P<0.05 (Mann-Whitney U).

FIG. 11: L-lactate-induced vasodilation is not altered in arteries from Kvβ1.1^{-/-} mice. Summary of L-lactate-induced dilation for arteries from Kvβ1.1^{-/-} and WT mice. n=6-7 arteries from 6-7 mice (two-way RM ANOVA).

FIGS. 12A-12B: Ablation of Kvβ proteins does not impact vasodilation in response to adenosine. (A) Representative diameter measurements obtained from mesenteric arteries (80 mmHg) from Kvβ1.1^{-/-} and Kvβ2^{-/-} mice and respective wild type (WT) control mice in the absence and presence of 1-100 μM adenosine. (B) Summary of adenosine-induced dilation in arteries from Kvβ1.1^{-/-} (left) and Kvβ2^{-/-} (right) versus respective WT mice. n=4-6 arteries from 3-6 mice; ns: P≥0.05 (two-way RM ANOVA).

FIG. 13. Heart rate and mean arterial pressure in double transgenic SM22α-rtTA:TRE-β1 and single transgenic control SM22α-rtTA mice in the absence and presence of catecholamine-induced stress. Summary graphs showing heart rate (HR; left) and mean arterial pressure (MAP; right) in SM22α-rtTA:TRE-β1 and SM22α-rtTA mice before and after intravenous infusion of norepinephrine (0-5 μg/kg·min⁻¹). n=5 each; ns: P≥0.05 (two-way ANOVA).

### DETAILED DESCRIPTION

Voltage-gated potassium (Kv) channels in vascular smooth muscle are essential for coupling myocardial blood flow (MBF) with the metabolic demand of the heart. These channels consist of a transmembrane pore domain that associates with auxiliary Kvβ1 and Kvβ2 proteins, which differentially regulate Kv function in excitable cells. Nonetheless, the physiological role of Kvβ proteins in regulating vascular tone and metabolic hyperemia in the heart has remained unknown.

The study tested the hypothesis that Kvβ proteins confer oxygen sensitivity to vascular tone and are required for regulating blood flow in the heart. Briefly, mice lacking Kvβ2 subunits exhibited suppressed MBF, impaired cardiac contractile performance, and failed to maintain elevated arterial blood pressure in response to catecholamine-induced stress. In contrast, ablation of Kvβ1.1 reduced cardiac workload, modestly elevated MBF, and preserved cardiac function during stress compared with wild type mice. Coronary arteries isolated from Kvβ2^{-/-}, but not Kvβ1.1^{-/-}, mice, had severely blunted vasodilation to hypoxia when compared with arteries from wild type mice. Moreover, vasodilation of small diameter coronary and mesenteric arteries due to L-lactate, a biochemical marker of reduced tissue oxygenation and anaerobic metabolism, was significantly attenuated in vessels isolated from Kvβ2^{-/-} mice. Inducible enhancement of the Kvβ1:Kvβ2 ratio in Kv1 channels of arterial smooth muscle abolished L-lactate-induced vasodilation and suppressed the relationship between MBF and cardiac workload.

In conclusion, the Kvβ proteins differentially regulate vascular tone and myocardial blood flow, whereby Kvβ2 promotes and Kvβ1.1 inhibits oxygen-dependent vasodilation and augments blood flow upon heightened metabolic demand.

Formulations and Methods of Administration

In certain embodiments, an effective amount of the therapeutic composition is administered to the subject. “Effective amount” or “therapeutically effective amount” are used interchangeably herein, and refer to an amount of a compound, formulation, material, or composition, as described herein effective to achieve a particular biological result.

In certain embodiments, the therapeutic composition is administered via intramuscular, intradermal, or subcutaneous delivery. In certain embodiments, therapeutic composition is administered via a mucosal surface, such as an oral, or intranasal surface. In certain embodiments, the therapeutic composition is administered via intrasternal injection, or by using infusion techniques.

In certain embodiments, “pharmaceutically acceptable” refers to those properties and/or substances which are acceptable to the patient from a pharmacological/toxicological point of view and to the manufacturing pharmaceutical chemist from a physical/chemical point of view regarding composition, formulation, stability, patient acceptance and bioavailability. “Pharmaceutically acceptable carrier” refers to a medium that does not interfere with the effectiveness of the biological activity of the active ingredient(s) and is not toxic to the host to which it is administered.

The compositions of the invention may be formulated as pharmaceutical compositions and administered to a mammalian host, such as a human patient, in a variety of forms adapted to the chosen route of administration, i.e., orally, intranasally, intradermally or parenterally, by intravenous, intramuscular, topical or subcutaneous routes.

Thus, the present compounds may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The active compound may also be administered intravenously or intraperitoneally by infusion or injection. Solutions of the active compound or its salts may be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient that are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions. For topical administration, the present compounds may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Additional ingredients such as fragrances or antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

Formulations will contain an effective amount of the active ingredient in a vehicle, the effective amount being readily determined by one skilled in the art. “Effective amount” is meant to indicate the quantity of a compound necessary or sufficient to realize a desired biologic effect. The active ingredient may typically range from about 1% to about 95% (w/w) of the composition, or even higher or lower if appropriate. The amount for any particular application can vary depending on such factors as the severity of the condition. The quantity to be administered depends upon factors such as the age, weight and physical condition of the animal considered for vaccination and kind of concurrent treatment, if any. The quantity also depends upon the capacity of the animal's immune system to synthesize antibodies, and the degree of protection desired. Typically, dosages used in vitro may provide useful guidance in the amounts useful for in situ administration of the composition, and animal models may be used to determine effective dosages for treatment of particular disorders. Various considerations are described, e.g., in Gilman et al., eds., Goodman And Gilman's: The Pharmacological Bases of Therapeutics, 8th ed., Pergamon Press, 1990; and Reminpton's Pharmaceutical Sciences, 17th ed., Mack Publishing Co., Easton, Pa., 1990, each of which is herein incorporated by reference. Additionally, effective dosages can be readily established by one of ordinary skill in the art through routine trials establishing dose response curves. The subject is immunized by administration of the composition thereof in one or more doses. Multiple doses may be administered as is required to maintain a state of immunity to the target. For example, the initial dose may be followed up with a booster dosage after a period of about four weeks to enhance the immunogenic response. Further booster dosages may also be administered. The composition may be administered multiple (e.g., 2, 3, 4 or 5) times at an interval of, e.g., about 1, 2, 3, 4, 5, 6 or 7, 14, or 21 days apart.

Intranasal formulations may include vehicles that neither cause irritation to the nasal mucosa nor significantly disturb ciliary function. Diluents such as water, aqueous saline or other known substances can be employed with the subject invention. The nasal formulations may also contain preservatives such as, but not limited to, chlorobutanol and benzalkonium chloride. A surfactant may be present to enhance absorption of the subject proteins by the nasal mucosa.

Oral liquid preparations may be in the form of, for example, aqueous or oily suspension, solutions, emulsions, syrups or elixirs, or may be presented dry in tablet form or a product for reconstitution with water or other suitable vehicle before use. Such liquid preparations may contain conventional additives such as suspending agents, emulsifying agents, non-aqueous vehicles (which may include edible oils), or preservative.

Thus, the present compositions may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the present compositions may be combined with one or more excipients and used in the form of ingestible tablets, buccal tablets, troches, capsules, elixirs, suspensions, syrups, wafers, and the like. Such preparations should contain at least 0.1% of the present composition. The percentage of the compositions may, of course, be varied and may conveniently be between about 2 to about 60% of the weight of a given unit dosage form. The amount of present composition in such therapeutically useful preparations is such that an effective dosage level will be obtained.

Useful dosages of the compositions of the present invention can be determined by comparing their in vitro activity, and in vivo activity in animal models. The amount of the compositions described herein required for use in treatment will vary with the route of administration and the age and condition of the subject and will be ultimately at the discretion of the attendant veterinarian or clinician.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

The invention will now be illustrated by the following non-limiting Example.

### Example 1

### Myocardial Blood Flow Control by Oxygen Sensing Vascular Kvβ Proteins

Voltage-gated potassium (Kv) channels expressed throughout the resistance vasculature regulate blood vessel diameter and control tissue perfusion. Whereas channels belonging to the Kv1 family are known to regulate blood flow in the heart, the molecular components that establish the metabolic sensitivity of the channel have not been identified. Our research has revealed a previously unknown physiological role for intracellular regulatory proteins that interact with the pore of the Kv channel (i.e., the Kv beta proteins). We have found that these proteins control the metabolic sensitivity of Kv1 channels in the coronary arterial network and could thereby serve as an important target for the modulation of blood flow to the heart.

In this study, we tested the hypothesis that regulation of myocardial blood flow (MBF) by Kv1 channels depends upon their auxiliary Kvβ subunits. The Kvβ proteins are functional aldo-keto reductases that bind NAD(P)(H) and differentially regulate channel gating in response to changes in cellular redox status. Hence, these proteins represent a plausible molecular link between metabolic activity, oxygen availability, and Kv activity that could regulate vasoreactivity. The mammalian genome encodes three Kvβ proteins, which have been shown to control the voltage sensitivity, surface localization, and subcellular distribution of Kv1 channels in excitable cells of the cardiovascular and nervous systems. Consistent with this, in our previous work, we reported that Kvβ proteins support the functional expression of Kv channels in cardiomyocytes and contribute to the metabolic regulation of cardiac repolarization. The Kvβ proteins are expressed throughout the coronary vasculature of humans and rodents, and we have recently reported that native Kv1 channels of coronary arterial myocytes are heteromeric assemblies of Kvβ1.1 and Kvβ2 proteins. Using a combination of genetically engineered mice with ex vivo and in vivo approaches, we now report that Kvβ1.1 and Kvβ2 have contrasting roles in regulating MBF and cardiac function under stress, and that they impart oxygen sensitivity to vascular tone.

Methods

Animals: All animal procedures were conducted as approved by the Institutional Animal Care and Use Committees at the University of Louisville and Northeast Ohio Medical University. Kvβ1.1^{-/-} and Kvβ2^{-/-} mice and strain-matched wild type (C57Bl/6N and 129/SvEv, respectively) mice (25-30 g body mass) were bred in house and fed normal rodent chow. Transgenic animals were generated (Cyagen) with mouse Kcnab1 (NM_01059734) at the control of the tetracycline responsive element (TRE, 2nd generation) promoter (TRE-Kcnab1.1). Hemizygous TRE-Kcnab1.1 mice were bred with transgenic mice with the reverse tetracycline transactivator under the control of the murine SM22-alpha (SM22α) promotor (SM22α-rtTA; Jackson Laboratories, stock no. 006875, FVB/N-Tg(Tagln-rtTA)ElJwst/J)40 to yield double hemizygous SM22α-rtTA:TRE-Kcnab1.1 and littermate single transgenic SM22α-rtTA controls. To avoid confounding results due to the effects of estrogen on vascular Kv channel expression, only male mice (aged 3-6 months) were used for this study. All animals were housed in a temperature-controlled room on a 12:12 light:dark cycle with ad libitum access to food and water. Summarized body weight and cardiac structural parameters from echocardiographic studies (see below) are shown in Table 1. Mice were euthanized by intraperitoneal injection of sodium pentobarbital (150 mg·kg−1) and thoracotomy, and tissues were excised immediately for ex vivo functional and biochemical assessments.

TABLE 1
Body weight and cardiac structural parameters for wild type and Kvβ-null mice.

| | | | | | | SM22α- |
|---|---|---|---|---|---|---|
| | Wild type | | Wild type | | SM22α- | rtTA:TRE- |
| Measurement | (C57B16N) | Kvβ1.1^{-/-} | (129SvEv) | Kvβ2^{-/-} | rtTA | Kvβ1 |
| Body Weight (g) | 26.7 ± 1.2 | 25.2 ± 2.0 | 25.8 ± 2.6 | 25.0 ± 0.7 | 27.6 ± 0.3 | 25.6 ± 0.8 |
| Wall Thickness | | | | | | |
| LVPWd (mm) | 1.06 ± 0.07 | 1.03 ± 0.05 | 1.29 ± 0.11 | 1.01 ± 0.06 | 1.15 ± 0.11 | 1.14 ± 0.15 |
| LVPWs (mm) | 1.57 ± 0.12 | 1.41 ± 0.04 | 1.62 ± 0.11 | 1.33 ± 0.07 | 1.61 ± 0.11 | 1.48 ± 0.16 |
| LVAWd (mm) | 0.97 ± 0.03 | 1.25 ± 0.02* | 1.23 ± 0.06 | 1.10 ± 0.04 | 1.15 ± 0.06 | 1.05 ± 0.10 |
| LVAWs (mm) | 1.43 ± 0.05 | 1.71 ± 0.03* | 1.67 ± 0.07 | 1.52 ± 0.04 | 1.77 ± 0.08 | 1.53 ± 0.15 |
| RWT | 0.59 ± 0.03 | 0.61 ± 0.06 | 0.75 ± 0.06 | 0.63 ± 0.04 | 0.69 ± 0.05 | 0.66 ± 0.06 |
| LV Mass (mg) | 106.7 ± 6.4 | 120.6 ± 4.5 | 142.0 ± 16.5 | 103.9 ± 6.1* | 120.2 ± 12.5 | 111.6 ± 12.7 |

Data are mean ± SEM.
*P < 0.05 vs. respective wild type/single transgenic control (unpaired t test).
Abbreviations: LVPWd, left ventricular posterior wall at diastole; LVPWs, left ventricular posterior wall at systole; LVAWd, left ventricular anterior wall at diastole; LVAWs, left ventricular anterior wall at systole; RWT, relative wall thickness; n = 3-8; *P < 0.05 (Mann Whitney U).

In vivo measurements of cardiac function and myocardial blood flow: Mice were anesthetized with 3% isoflurane and supplemental O₂, administered (1 L·min⁻¹) in a small induction chamber. After induction, the mice were placed on a controlled heating table in a supine position. Anesthesia was maintained throughout the procedure by delivery of 1-2% isoflurane and supplemental O₂ (0.5 L·min⁻¹). The extremities were secured to the surgical table by tape and a lubricated probe was inserted rectally to monitor body temperature. The chest, neck, and hind limb hair were then removed using a depilatory agent, the skin was rinsed with warm water, and the neck area was disinfected with 70% ethanol/betadine and an incision (10-15 mm) was made at the right side of the neck. For infusion of contrast agent and drugs, the jugular vein was isolated using blunt forceps and catheterized with sterilized PE-50 polyethylene tubing (pre-filled with heparinized saline; 50 U·ml⁻¹). The jugular vein catheter was then secured in place with two sutures. For continuous measurement of arterial blood pressure, a small incision was made on the hind limb and the femoral artery was isolated and cannulated with a 1.2 F pressure catheter (SciSense, Transonic Systems, Inc., Ithaca, NY, USA) connected to a PowerLab data acquisition system (ADInstruments, Colorado Springs, CO, USA) through a SP200 pressure interface unit designed to measure arterial blood pressure and heart rate. After cannulation, the pressure catheter was advanced −10 mm into the abdominal aorta.

Ultrasound gel was centrifuged in a 60 mL syringe (1500×g, 10 min) to remove air bubbles, warmed to 37° C., and applied to the chest. Cardiac function was measured by M-mode transthoracic echocardiographic imaging of the parasternal short axis view, mid-papillary level using a Vevo 2100 high resolution echocardiography imaging system (FujiFilm VisualSonics, Toronto, ON, Canada). Contrast echocardiography was performed by using Siemens ultrasound imaging system (Sequoia Acuson C512; Siemens Medical Systems USA Inc., Mountain View, CA) with a high-frequency linear-array probe (15L8) held in place by a 3D railing system. For myocardial contrast echocardiography (MCE), we administered lipid-shelled microbubbles, which were freshly prepared by sonication of a decafluorobutane gas-saturated aqueous suspension of distearoylphosphatidylcholine (2 mg/mL) and polyoxyethylene-40-stearate (1 mg/mL). The contrast agent was intravenously infused via the jugular vein catheter at a rate of ˜5×10⁵ microbubbles·min−1 (20 μl·min⁻¹) and MCE was performed by administering a multi-pulse contrast-specific pulse sequence to detect non-linear microbubble contrast signal at low mechanical index (MI=0.18-0.25). Data were acquired during and after a 1.9 MI pulse sequence to destruct microbubbles within the acoustic field, followed by imaging of replenished contrast signal.

Long axis images were obtained for perfusion imaging. All settings for processing were adapted and optimized for each animal: penetration depth was ˜2-2.5 cm, near field was focused on the middle of the left ventricle (long axis view), and gains were adjusted to obtain images with no signal from the myocardium and then held constant. Regions of interest (ROI) were positioned within the anterolateral region in the short axis view. A curve of signal intensity over time was obtained in the ROI and fitted to an exponential function: y=A(1−e^{-β4}), where y is the signal intensity at any given time, A is the signal intensity corresponding to the microvascular cross sectional volume, and β is the initial slope of the curve, which corresponds to the blood volume exchange frequency. Relative blood volume (RBV) was calculated as the ratio of myocardial to cavity signal intensity (RBV=A/ALV). ALV corresponds to the signal intensity for the LV cavity. Color coded parametric images were used to outline a region of interest (region of the left ventricle). Myocardial blood flow (MBF) was estimated as the product of RBV×β. The analysis of nearby regions within the myocardium and the left ventricle is proposed to compensate for regional beam inhomogeneities and contrast shadowing. MBF was calculated from the blood volume pool relative to the surrounding myocardial tissue, the exchange frequency (initial slope of curve), and tissue density ρ (ρ_{T}=1.05). MBF was measured in 3-5 different images obtained from the same condition (baseline and treatments). To compare the relationships between MBF and cardiac workload, a simple linear regression equation was fit to the data (Graphpad Prism 8). MCE analyses were performed by readers blinded to genotype and treatment.

Measurements of cardiac function, myocardial perfusion, and arterial blood pressure were performed at baseline, after administration of hexamethonium (5 mg·kg⁻¹, i.v.), and following successive doses of norepinephrine (0.5, 1.0, 2.5, and 5.0 μg·kg⁻¹·min⁻¹; ˜3 min duration each dose, followed by 3-5 min washout). Animals that did not complete the entire procedure of norepinephrine infusions (1-3 mice per group) were excluded from analysis. All data analyses and calculations of cardiac workload (double product of mean arterial pressure and heart rate), cardiac function, myocardial blood flow, and mean arterial pressure were performed offline. For pressure measurements, we used Lab Chart 8 software (ADInstruments, Colorado Springs, CO, USA). Left ventricular volume at end diastole (LVEDV) and end systole (LVESV), as well left ventricular internal diameter at end diastole (LVID,d) and end systole (LVID,s) were measured at steady state after drug infusions. Left ventricular volume was calculated by a modified Teichholz formula: LVV=((7.0/(2.4+LVID))*LVID³. Left ventricular ejection fraction (LVEF %) was calculated by: (LVEDV-LVESV)/LVEDV. All echocardiographic calculations and measurements were carried out offline using VevoLab 3.1 software (FujiFilm VisualSonics, Toronto, ON, Canada). All measurements were averaged over 3-5 cardiac cycles.

TABLE 2
Echocardiographic parameters in wild type and Kvβ-null mice.

| | | | | | | | SM22α- |
|---|---|---|---|---|---|---|---|
| | | Wild type | | Wild type | | SM22α- | rtTA:TRE- |
| Endocardial values | | (C57B16N) | Kvβ1.1^{-/-} | (129SvEv) | Kvβ2^{-/-} | rtTA | Kvβ1 |
| EDV (μl) | | | | | | | |
| NE (μg/kg · min⁻¹) | 0 | 61.7 ± 2.9 | 53.8 ± 8.1 | 53.9 ± 4.5 | 50.8 ± 2.3 | 44.6 ± 3.3 | 52.2 ± 1.5 |
| | 0.5 | 57.8 ± 2.3 | 53.5 ± 4.9 | 50.5 ± 7.8 | 48.3 ± 2.7 | 43.6 ± 2.9 | 45.9 ± 3.8 |
| | 1 | 61.6 ± 3.2 | 47.6 ± 8.1 | 46.7 ± 7.4 | 48.7 ± 4.6 | 49.4 ± 2.9 | 46.9 ± 4.9 |
| | 2.5 | 62.8 ± 3.7 | 41.5 ± 8.8 | 48.8 ± 6.0 | 52.8 ± 3.5 | 51.5 ± 2.8 | 47.5 ± 5.5 |
| | 5.0 | 66.1 ± 4.4 | 45.7 ± 11.9 | 56.4 ± 5.0 | 54.6 ± 4.5 | 56.9 ± 3.9 | 49.3 ± 5.7 |
| ESV (μl) | | | | | | | |
| NE (μg/kg · min⁻¹) | 0 | 26.14 ± 2.5 | 23.1 ± 4.6 | 26.3 ± 4.7 | 19.3 ± 2.5 | 13.7 ± 2.4 | 30.2 ± 3.5* |
| | 0.5 | 16.37 ± 1.4 | 21.3 ± 3.9 | 19.4 ± 3.7 | 13.6 ± 1.6 | 8.1 ± 2.0 | 17.7 ± 3.6 |
| | 1 | 17.12 ± 1.3 | 14.1 ± 3.4 | 14.7 ± 4.3 | 13.3 ± 1.8 | 10.0 ± 2.6 | 12.7 ± 3.2 |
| | 2.5 | 17.22 ± 1.7 | 9.4 ± 2.7 | 16.0 ± 2.7 | 15.7 ± 1.6 | 12.2 ± 3.1 | 13.2 ± 4.3 |
| | 5.0 | 17.85 ± 2.3 | 10.4 ± 3.4 | 17.3 ± 1.6 | 18.5 ± 3.1 | 13.4 ± 3.9 | 15.6 ± 3.9 |
| SV (μl) | | | | | | | |
| NE (μg/kg · min⁻¹) | 0 | 35.56 ± 2.4 | 30.7 ± 3.5 | 27.6 ± 0.9 | 31.5 ± 2.0 | 30.9 ± 1.9 | 22.0 ± 3.3 |
| | 0.5 | 41.46 ± 2.3 | 32.2 ± 1.8 | 31.2 ± 4.2 | 34.7 ± 2.4 | 35.4 ± 1.6 | 28.2 ± 2.7 |
| | 1 | 44.52 ± 2.3 | 33.5 ± 5.2 | 32.0 ± 3.7 | 42.7 ± 8.2 | 39.4 ± 1.8 | 34.2 ± 2.0 |
| | 2.5 | 45.63 ± 2.6 | 32.1 ± 6.1 | 32.8 ± 3.6 | 37.1 ± 2.8 | 39.3 ± 2.1 | 34.3 ± 1.4 |
| | 5.0 | 48.26 ± 2.7 | 35.3 ± 8.5 | 39.1 ± 4.1 | 36.1 ± 2.3 | 43.5 ± 3.1 | 33.7 ± 2.6 |
| EF (%) | | | | | | | |
| NE (μg/kg · min⁻¹) | 0 | 57.84 ± 3.2 | 57.8 ± 2.6 | 52.1 ± 4.6 | 62.4 ± 3.8 | 70.0 ± 3.8 | 42.3 ± 6.6 |
| | 0.5 | 71.64 ± 2.2 | 61.4 ± 4.2 | 63.5 ± 2.5 | 71.9 ± 3.3 | 82.3 ± 3.3 | 62.7 ± 6.0 |
| | 1 | 72.40 ± 1.2 | 70.9 ± 3.1 | 70.7 ± 5.3 | 73.1 ± 1.8 | 80.6 ± 4.2 | 74.2 ± 3.9 |
| | 2.5 | 72.88 ± 1.8 | 78.2 ± 2.0 | 67.9 ± 2.7 | 70.1 ± 2.6 | 77.4 ± 5.1 | 74.2 ± 5.7 |
| | 5.0 | 73.65 ± 2.4 | 79.1 ± 2.7 | 69.1 ± 2.4 | 67.5 ± 3.8 | 77.7 ± 5.8 | 69.9 ± 5.0 |
| FS (%) | | | | | | | |
| NE (μg/kg · min⁻¹) | 0 | 30.17 ± 2.1 | 29.8 ± 1.6 | 26.1 ± 2.8 | 33.7 ± 2.7 | 39.0 ± 3.2 | 20.5 ± 3.6* |
| | 0.5 | 40.48 ± 2.0 | 32.5 ± 2.8 | 34.2 ± 1.9 | 40.7 ± 2.8 | 50.9 ± 3.6 | 33.9 ± 4.1 |
| | 1 | 40.97 ± 1.0 | 40.1 ± 2.4 | 40.1 ± 4.6 | 41.4 ± 1.6 | 49.6 ± 4.3 | 42.7 ± 3.3 |
| | 2.5 | 41.47 ± 1.5 | 46.0 ± 1.8 | 37.4 ± 2.1 | 39.0 ± 2.2 | 46.8 ± 4.8 | 43.1 ± 4.8 |
| | 5.0 | 42.39 ± 2.1 | 47.1 ± 2.4 | 38.3 ± 1.9 | 37.3 ± 3.0 | 48.1 ± 6.0 | 39.3 ± 4.2 |

Data are mean ± SEM.
*P < 0.05; (mixed effects with Tukey post hoc test).
Abbreviations: EDV, left ventricular end diastolic volume; ESV, left ventricular end systolic volume; SV, stroke volume; EF, ejection fraction; FS, fractional shortening.
n = 3-7; *P < 0.05 (mixed effects).

Arterial diameter measurements: Primary and secondary branches of the left anterior descending coronary arteries and third and fourth order branches of mesenteric arteries and were dissected and kept in ice-cold isolation buffer consisting of (in mM): 134 NaCl, 6 KCl, 1 MgCl₂, 2 CaCl₂, 10 HEPES, 7 D-glucose, pH 7.4. Isolated arteries were used for arterial diameter measurements within 8 h after dissection. Isolated arteries were cleaned of connective tissue and cannulated on glass micropipettes mounted in a linear alignment single vessel myograph chamber (Living Systems Instrumentation, St. Albans, VT, USA). For some experiments, the vascular endothelium was functionally ablated by passage of air through the lumen (˜30 s) during the cannulation procedure. After cannulation, the chamber was placed on an inverted microscope and arteries were equilibrated at 37° C. and intravascular pressure of 20 mmHg, maintained with a pressure servo control unit (Living Systems Instrumentation, St. Albans, VT, USA) under continuous perfusion (3-5 ml·min⁻¹) of physiological saline solution (PSS) consisting of (in mM): 119 NaCl, 4.7 KCl, 1.2 KH₂PO₄, 1.2 MgCl₂, 7 D-glucose, 24 NaHCO₃, 2 CaCl₂, maintained at pH 7.35-7.45 via aeration with gas mixture containing 5% CO₂ and 20% O₂ (balanced with N₂).

Following an equilibration period (45-60 min), lumenal diameter was continuously monitored and recorded with a charge coupled device (CCD) camera and edge detection software (IonOptix, Milton, MA, USA). Experiments were performed to examine effects of step-wise increases in intravascular pressure (20-100 mmHg), elevated [K⁺]ₒ (via isosmotic replacement of KCl for NaCl), the synthetic thromboxane A₂ analogue U46619 (Tocris Bioscience, Minneapolis, MN, USA), adenosine (Sigma Aldrich, St. Louis, MO, USA), or L-lactate (Sigma Aldrich, St. Louis, MO, USA). For some experiments, hypoxic bath conditions were generated by perfusion of 1 mM Na₂S2O₄-containing PSS aerated with 5% CO₂ (balance N₂; 0% O₂). Bath O₂ levels were measured using a dissolved oxygen meter (World Precision Instruments, Sarasota, FL, USA). At the end of each experiment, the maximum passive diameter was measured in the presence of Ca²⁺-free PSS containing the L-type Ca²⁺ channel inhibitor nifedipine (1 μM) and adenylyl cyclase activator forskolin (0.5 μM), as described previously. Vasoconstriction is expressed as a decrease in arterial diameter relative to the maximum passive diameter at a given intravascular pressure. Changes in diameter (e.g., vasodilation) are normalized to differences from baseline and maximum passive diameters for each experiment.

Western blotting: Whole tissue lysates were obtained from mesenteric arteries and brain, as described previously. Briefly, tissues were homogenized in lysis buffer containing 150 mM NaCl, 50 mM Tris-HCl, 0.25% deoxycholic acid, 1% NP-40, 1 EDTA, with protease inhibitors (Complete Mini protease inhibitor cocktail, Roche) and phosphatase inhibitors (Phosphatase Inhibitor Cocktail, Thermo), pH 7.4. Homogenates were sonicated and centrifuged at 10,000×g (10 min, 4° C.), and supernatants were boiled in Laemmli sample buffer for 10 min and run on a 4-20% Mini-PROTEAN TGX precast Protein gel (Bio-Rad) and subjected to SDS-PAGE. Following transfer to a polyvinylidene fluoride (PVDF) membrane, total protein was assessed for each lane by staining with Ponceau S. Non-specific binding was blocked with 5% dry milk in Tris-buffered saline (TBS) and membranes were then incubated overnight (at 4° C.) in primary antibodies against Kvβ1.1 (Neuromab, 75-018, 1:500) in TBS containing 0.1% Tween-20 (TBS-t). After washing (5× with TBS-t at room temperature), the membranes were incubated in TB S-t containing 5% dry milk and horseradish peroxidase (HRP)-conjugated secondary antibodies (anti-mouse IgG; Cell Signaling, 7076S, 1:3000). HRP was detected with Pierce ECL Plus Western Blotting Substrate (Thermo) and a myECL imaging system (Thermo). Densitometry was performed for immunoreactive bands using FIJI software (National Institutes of Health).

In situ proximity ligation: Arterial myocytes were isolated from coronary and mesenteric arteries using enzymatic digestion procedures, similar to those described previously. Briefly, arteries were incubated in digestion buffer containing (in mM): 140 NaCl, 5 KCl, 2 MgCl₂, 10 HEPES, 10 glucose, pH 7.4 at 37° C. for 1 min. The buffer was exchanged for digestion buffer containing papain (1 mg/mL; Worthington) and dithiothreitol (1 mg/mL; Sigma Aldrich) and incubated at 37° C. for 5 min with gentle agitation; the papain/dithiothreitol buffer was then exchanged for digestion buffer containing collagenase type H (1.25 mg/mL; Sigma Aldrich) and trypsin inhibitor (1 mg/mL; Sigma Aldrich) and incubated at 37° C. for 5 min with gentle agitation. The digested tissue was then washed three times with ice-cold enzyme-free digestion buffer and triturated with a flame-polished Pasteur pipette to liberate individual arterial myocytes.

Isolated arterial myocytes were transferred in suspension to glass microscope slides and allowed to adhere (˜20 min; room temperature). After adherence, the cells were washed with phosphate-buffered saline (PBS) and fixed in paraformaldehyde (4% in PBS) (for 10 min at room temperature). Following fixation, cells were permeabilized in PBS containing 0.1% Triton X-100 (for 10 min at room temperature). To detect protein-protein proximity (≤40 nm), an in situ proximity ligation assay (PLA) kit (Duolink; Sigma Aldrich) was used per manufacturer's instructions. Cells were blocked with Duolink blocking solution and incubated in primary antibodies against Kv1.5 (Neuromab, 75-011, 1:50), Kvβ1 (Abcam, AB174508, 1:100), and Kvβ2 (Aviva system biology, ARP37678_T100, 1:100). Antibody-labelled Kv subunits were detected with oligonucleotide-conjugated PLA probe secondary antibodies (anti-rabbit PLUS and anti-mouse MINUS) followed by a solution with PLA probe-specific oligonucleotides and ligase to generate circular nucleotide products at sites of probe-probe proximity. Cells were then incubated (100 min, 37° C.) in a solution consisting of polymerase and fluorophore-tagged oligonucleotides for rolling circle amplification, concatemeric product generation, and fluorescent labelling. After washing, the slides were mounted with Duolink mounting media containing DAPI nuclear stain and coverslips were sealed with nail polish. Fluorescent images were captured using a Keyence BZ-X800 All-in-One fluorescence microscopy imaging system. Images were analyzed to obtain counts of total fluorescent PLA punctae in each cell using FIJI software (National Institutes of Health). Images from complete z-series (1 μm step) for each cell were flattened using the z-project function and PLA-associated punctate particles for each cell were counted and normalized to the area of the cell footprint, obtained from transmitted light images.

Patch clamp electrophysiology: Arterial myocytes were isolated from coronary arteries as described above. Isolated arterial myocytes were allowed to adhere (5 min) to a glass coverslip in a recording chamber. Total outward K⁺ currents (IK) were recorded from coronary arterial myocytes using the perforated whole cell configuration of the patch clamp technique in voltage clamp mode using an Axopatch 200B patch clamp amplifier (Axon Instruments). Borosilicate glass pipettes were pulled to a resistance of 5-7 MΩ and filled with a solution containing (in mM) 87 K⁺-aspartate, 20 KCl, 1 MgCl₂, 5 Mg²⁺ ATP, 10 EGTA, and 10 HEPES with 36 μg/mL amphotericin B (pH 7.2 with KOH). Cells were bathed in external solution containing (in mM) 134 NaCl, 6 KCl, 1 MgCl₂, 0.1 CaCl₂, 10 glucose, and 10 HEPES (pH 7.4 with NaOH). K⁺ currents were recorded from each cell in the absence and presence of L-lactate (10 mM) in bath solution with and without psora-4 (500 nM). To obtain the I-V relationships, cells were sequentially depolarized for 500 ms from a holding potential of −70 mV to +50 mV in 10 mV increments. All patch clamp experiments were performed at ambient room temperature (21-23° C.). Patch clamp data were analyzed suing Clampfit 9 software (Axon Instruments). IK is expressed as peak currents reached during the period of depolarization normalized by cell capacitance and expressed as pA/pF.

Statistics: Data are shown as means±SEM unless otherwise indicated. All data were analyzed using GraphPad Prism 9 (GraphPad Software). Detailed statistics, including normality, comparisons, tests, and post-hoc tests, exact P values, and n values can be found in the Statistics Supplement. Normality was determined by Shapiro-Wilk test. Unpaired or paired t tests were performed to compare two groups with normally distributed datasets. One-way ANOVA was used to compare three or more groups with normally distributed datasets and post-hoc tests were used for multiple comparisons as indicated in the supplemental tables. Two-way repeated measures ANOVA was performed to test for differences in time and genotype or treatment. For datasets that did not pass normality testing, appropriate nonparametric tests were used (Mann Whitney U, Friedman). No corrections were made for multiple testing across experiments throughout the study. P<0.05 was considered statistically significant. Representative data that are displayed in figures were selected based on accurate representation of groups means.

Results

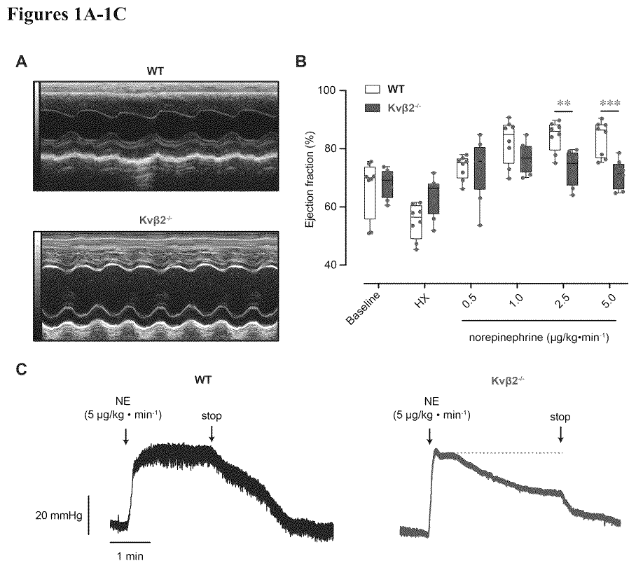

Kvβ2 is required for sustained cardiac pump function during stress. Under conditions of heightened cardiac workload, sustained pump function is critically dependent on Kv1-mediated coronary vasodilation for sufficient oxygen delivery to meet myocardial metabolic demand. We first tested whether loss of Kvβ proteins affects cardiac performance under stress. FIG. 1A shows representative M mode echocardiographic images from wild type (WT) and Kvβ2^{-/-} animals during intravenous infusion of norepinephrine (5 μg/kg·min⁻¹). Norepinephrine enhanced cardiac function, as indicated by an increase in ejection fraction. However, steady-state ejection fraction during infusion of 2.5 and 5 μg/kg·min⁻¹ norepinephrine was significantly lower in Kvβ2^{-/-} animals than in WT animals (FIG. 1A, B). Specifically, ejection fraction after ˜1 min of 5 μg/kg·min⁻¹ norepinephrine infusion was 71±1.7% in Kvβ2^{-/-} mice versus 84±2.2% in WT animals. Ejection fraction in Kvβ1.1^{-/-} mice did not differ significantly from that in WT mice at any dose of norepinephrine (P=0.093).

FIG. 1C shows exemplary effects of norepinephrine infusion on arterial blood pressure in WT and Kvβ2^{-/-} mice. Norepinephrine infusion increased steady state blood pressure in both groups. Consistent with our previous report, norepinephrine led to an increase in arterial blood pressure in WT animals that was sustained for the duration of drug administration. However, in Kvβ2^{-/-} mice, norepinephrine-induced elevation of pressure was not sustained, but declined after ˜40 s of infusion. This inability to maintain elevated blood pressure during stress is reminiscent of effects in Kv1.5-null mice. Therefore, as is the case with Kv1.5, Kvβ2 appears to play an essential role in supporting cardiac contractile performance under conditions of catecholamine stress and enhanced cardiac workload.

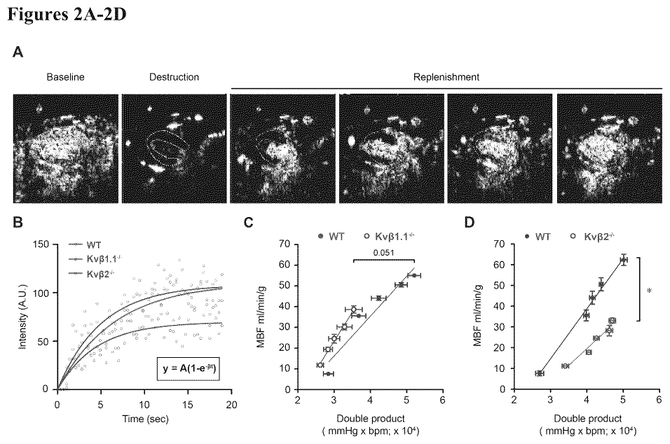

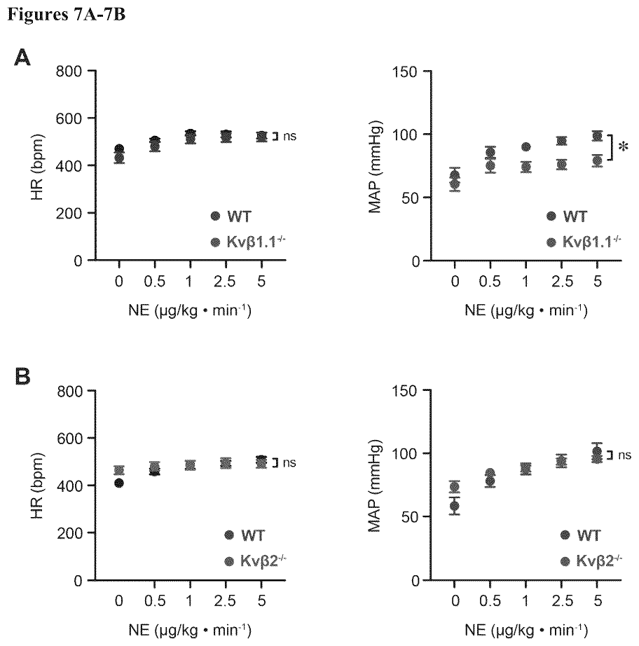

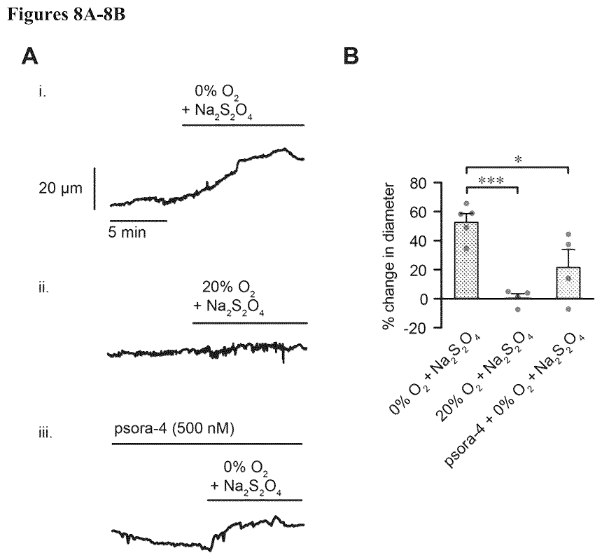

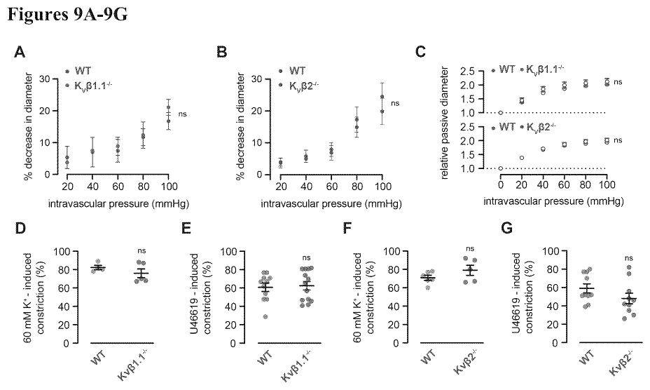

Relationship between myocardial blood flow and cardiac workload is disrupted in Kvβ2-null mice. The inability of Kvβ2^{-/-} mice to sustain cardiac performance may reflect insufficient oxygen delivery during stress. Thus, we postulated that Kvβ proteins may be integral to the relationship between myocardial blood flow (MBF) and cardiac workload. To test this, we used myocardial contrast echocardiography (MCE)^{11, 12} to compare MBF in WT and Kvβ-null mice. MCE uses high-power ultrasound to destruct lipid-shelled echogenic microbubbles in circulation. Subsequent replenishment of signal intensity in a region of interest following disruption is used to calculate the tissue perfusion (FIG. 2A, see Methods). Because MBF responds to changes in ventricular workload and myocardial metabolic activity, we used MCE to evaluate MBF as a function of cardiac workload (i.e., double product of mean arterial blood pressure×heart rate),¹² monitored at baseline and during intermittent intravenous infusions of norepinephrine (0.5-5 μg/kg·min⁻¹). FIG. 2B shows representative contrast signal intensities plotted over a period of ˜10 s after microbubble destruction and fit with a one-phase exponential function (see inset) in WT (129SvEv), Kvβ1.1^{-/-}, and Kvβ2^{-/-} mice (5 μg/kg·min⁻¹ norepinephrine). The relationship between MBF and double product shows a modest elevation of MBF, albeit across a lower workload range in Kvβ1.1^{-/-} mice compared with WT mice (FIG. 2C). However, consistent with impaired cardiac function under stress conditions described above (see FIG. 1), levels of MBF recorded in Kvβ2^{-/-} mice were markedly reduced. Specifically, linear regression analysis showed a significant reduction in the slope of the MBF-work relationship in Kvβ2^{-/-} mice (FIG. 2D). MAP, HR, and echocardiographic data at baseline and after acute norepinephrine infusion for each group are summarized in FIG. 7 and Table 1. Note that cardiac workload in Kvβ1.1^{-/-} mice was reduced due to lower MAP relative to corresponding wild type mice in the presence of 1-5 μg/kg·min⁻¹ norepinephrine (see FIG. 8C and FIG. 1). However, MAP, HR, and double product were not significantly different between WT and Kvβ2^{-/-} mice over the tested range of norepinephrine. Taken together, these data reflect differential roles for Kvβ1.1 and Kvβ2 proteins in regulating MBF, whereby loss of Kvβ2 suppresses MBF and impairs cardiac function as the heart is subjected to increased workloads.

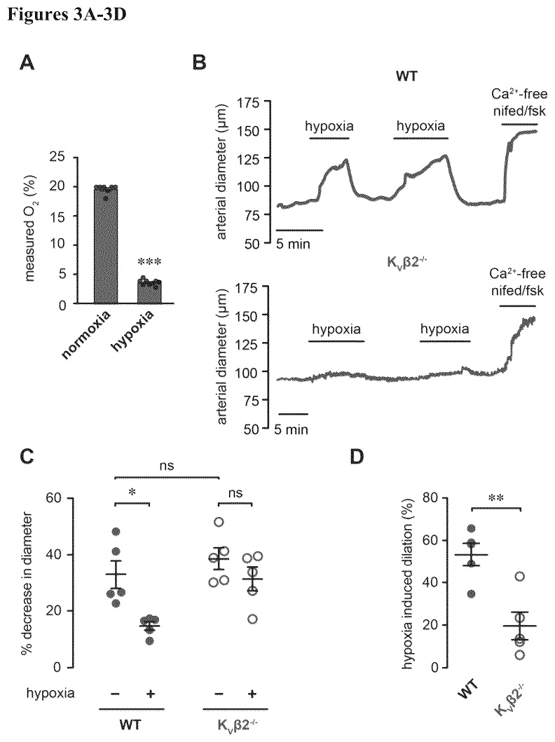

Oxygen sensitivity of coronary arterial diameter is modified by Kvβ2. Impaired Kv1-mediated coronary vasodilation results in a markedly reduced myocardial oxygen tension during increased metabolic demand. 22 We therefore posited that coronary vasodilation in response to metabolic stress may be impaired by loss of Kvβ2. Arteries of the systemic circulation exhibit robust dilation in response to metabolic stressors such as hypoxia and intracellular acidosis via a number of purported mechanisms, including activation of Kv channels. Hence, we examined the ex vivo vasoreactivity of coronary arteries isolated from WT and Kvβ2^{-/-} mice in response to an acute reduction in oxygen. When subjected to physiological intravascular pressures, isolated coronary arteries developed myogenic tone (i.e., 8±2% and 11±2% at 60 and 80 mmHg, respectively). To evaluate vasodilatory capacity, arteries were pressurized (60 mmHg), pre-constricted with 100 nM U46619, and subjected to hypoxic bath conditions (physiological saline solution aerated with 95% N₂/5% CO₂ and containing 1 mM hydrosulfite). Direct measurement of bath O₂ levels confirmed a significant reduction in O₂ from control levels during application of hypoxic conditions (FIG. 3A). As shown in FIG. 3B (top) and FIG. 8, coronary arteries isolated from WT mice responded to hypoxic perfusate with robust and reversible dilation. Vasodilation was not observed when 1 mM hydrosulfite was applied in the presence of 20% O₂ (FIG. 8). Consistent with the involvement of Kv1 channels, the selective Kv1 inhibitor psora-4 (500 nM) significantly attenuated (˜58%) hypoxia-induced vasodilation (FIG. 8). Likewise, hypoxia-induced dilation was significantly reduced in arteries from Kvβ2^{-/-} mice (19.6±6.4%) compared with arteries from WT mice (56.9±6.2%) (FIG. 3B-D). Together, these data suggest that Kvβ2 proteins facilitate vasodilation to reduced P02 and support the notion that Kvβ proteins link tissue perfusion to local oxygen consumption.

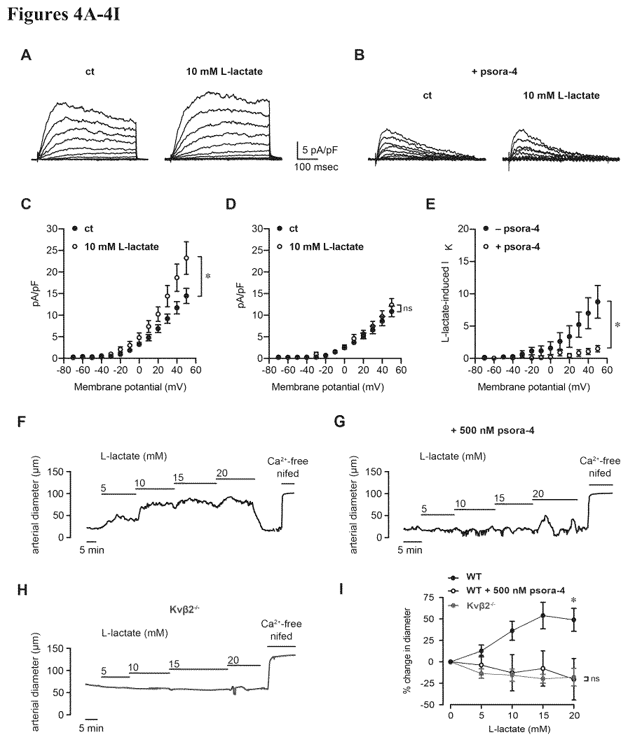

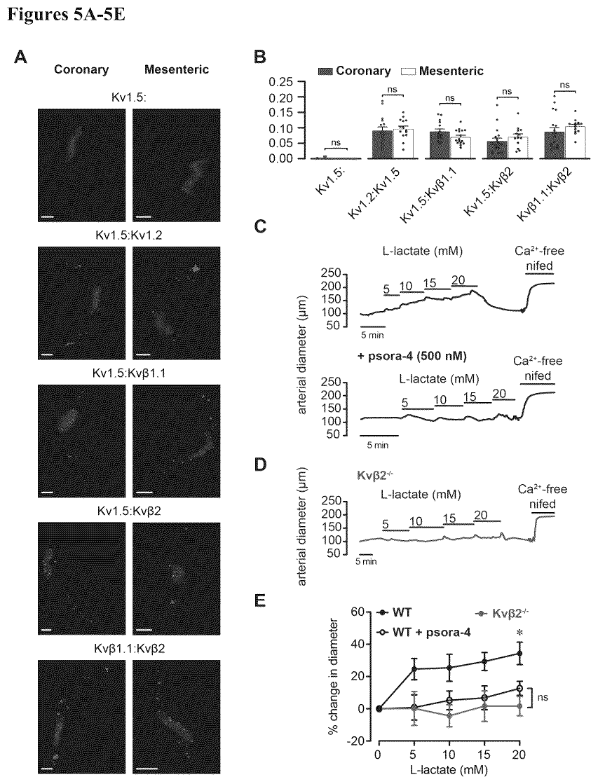

L-lactate augments I_{Kv} in coronary arterial myocytes and induces coronary vasodilation via Kvβ2. We tested whether Kv1 activity in coronary arterial myocytes is sensitive to acute changes in oxygen due to alterations in cellular redox potential via elevation of L-lactate. Our reasoning for examining the effects of L-lactate was two-fold: first, myocardial underperfusion leads to a rapid decline in tissue P02, increased anaerobic metabolism, and net accumulation of L-lactate that can promote feedback coronary vasodilation to increase MBF.^{21, 28-31} Second, it is plausible that Kv1 channels, via association with Kvβ proteins, may be acutely responsive to changes in lactate secondary to modification of cellular NADH:NAD⁺ ratio after uptake and interconversion to pyruvate via the lactate dehydrogenase reaction.^{15, 17, 32-35} Consistent with this expectation, using the perforated whole cell configuration of the patch clamp technique, we observed a significant increase in outward K⁺ current density (pA/pF) in isolated coronary arterial myocytes immediately following (1-3 min) application of 10 mM L-lactate in the bath (FIG. 4A, C). However, this effect was abolished when L-lactate was applied in the presence of the Kv1 blocker psora-4 (500 nM, FIG. 4B, D). The change in IK induced by application of 10 mM L-lactate in coronary arterial myocytes in the absence and presence of psora-4 is shown in FIG. 4E. These data indicate that L-lactate acutely potentiates I_{KV} in coronary arterial myocytes.

We next examined the vasodilatory response of preconstricted coronary arteries to increasing concentrations of extracellular L-lactate. As shown in FIG. 4F and consistent with previous studies, isolated coronary arteries that were pre-constricted with 100 nM U46619 exhibited step-wise vasodilation in response to elevation of external L-lactate (5-20 mM). This effect was abolished when L-lactate was applied in the presence of 500 nM psora-4 (FIG. 4G, I), consistent with involvement of I_{Kv} described above. Furthermore, L-lactate-induced vasodilation was also abolished in arteries isolated from Kvβ2^{-/-} mice, indicating a key role for this subunit in L-lactate-induced vasodilation (FIG. 411, I). These data are consistent with the notion that the regulation of Kvβ2 via vascular intermediary metabolism controls coronary vasodilatory function upon acute changes in myocardial oxygen tension.

Functional role for Kvβ2 in L-lactate-induced vasodilation of resistance mesenteric arteries. We next asked whether the role for Kvβ in redox-dependent vasoreactivity is confined to the coronary vasculature or is generally observed in peripheral resistance arterial beds where Kv1 prominently controls vascular tone. For this, we first compared Kvβ protein-protein interactions in arterial myocytes of coronary versus mesenteric (3^{rd} and 4^{th} order) arteries using in situ proximity ligation (PLA), as previously described. The PLA method is based on dual labelling of proteins that are located within close proximity (<40 nm), and thus, is an approach used to identify protein-protein interactions in complexes with molecular resolution. We observed robust PLA-associated fluorescent signals in coronary arterial myocytes that were co-labelled with Kv1.5 and Kv1.2, Kv1.5 and Kvβ1, Kv1.5 and Kvβ2, or Kvβ1 and Kvβ2 (FIG. 5A), consistent with heteromeric oligomerization of Shaker channels. The number of fluorescent sites assigned to these—α/α, α/β, and β/β interactions were similar between coronary and mesenteric arterial myocytes (FIG. 5A, B). PLA-associated fluorescence in cells labeled for Kv1.5 alone was negligible for arterial myocytes of both beds. These data suggest that Kv α/β subunit expression patterns and interactions are similar in arterial myocytes of these two distinct vascular beds.

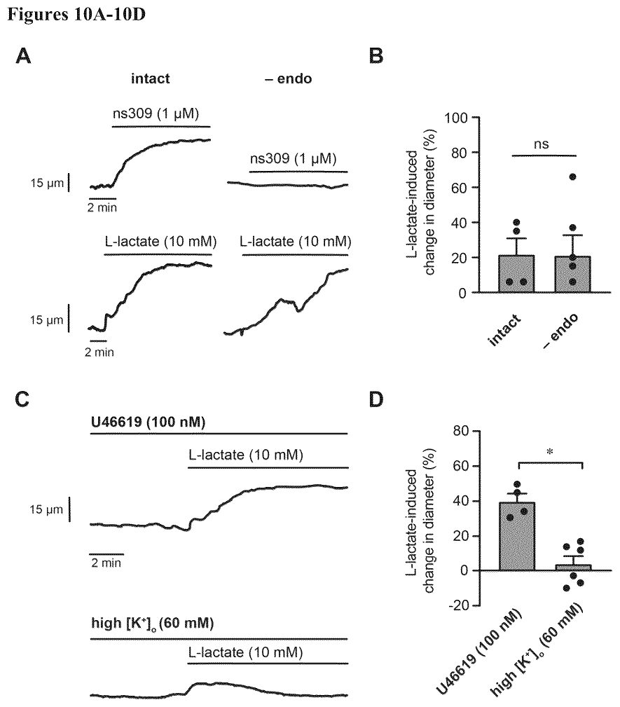

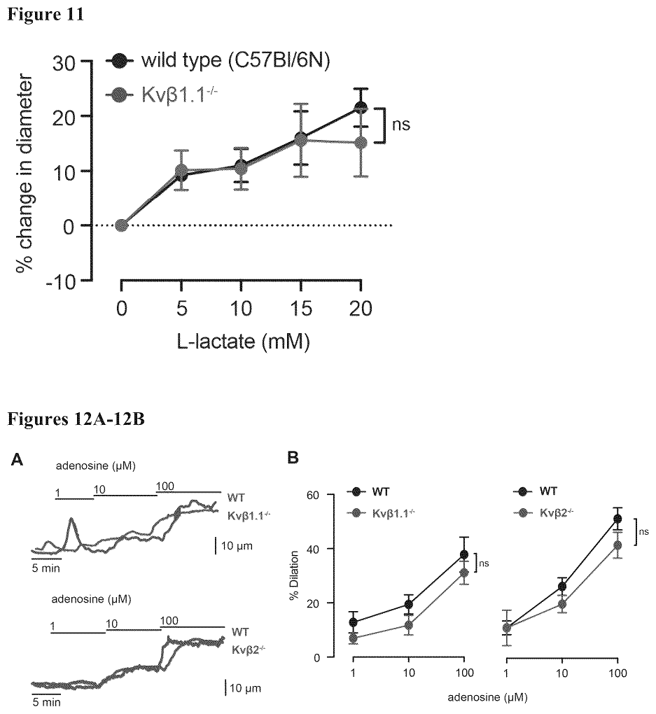

Next, we tested whether knockout of Kvβ1.1 or Kvβ2 alters the regulation of mesenteric arterial diameter. Note that ablation of either of these Kvβ proteins had no statistically significant effect on the active (i.e., myogenic tone) or passive responses to increases in intravascular pressure, nor did it impact vasoconstriction responses to direct membrane potential depolarization with 60 mM K⁺ or the stable thromboxane A₂ receptor agonist U46619 (100 nM; FIG. 9). Similar to observations in isolated coronary arteries (see FIG. 4F), application of L-lactate (5-20 mM) resulted in robust and reversible dilation of isolated mesenteric arteries (FIG. 5C). L-lactate-mediated vasodilation was insensitive to endothelial denudation but was abolished when arteries were constricted with elevated external K⁺, rather than U46619 (FIG. 10). Consistent with observations in isolated coronary arteries, vasodilation in response to L-lactate was eliminated by the Kv1-selective inhibitor psora-4 and loss of Kvβ2 (FIG. 5C-E). The dilatory response to L-lactate was not significantly different between arteries from Kvβ1.1^{-/-} mice when compared with arteries from corresponding WT animals (FIG. 11). Moreover, in contrast to the disparate effects of L-lactate, vasodilation induced by adenosine (1-100 μM) was not significantly different between Kvβ1.1^{-/-} or Kvβ2^{-/-} arteries, when compared with corresponding WT arterial preparations (FIG. 12). Together with results shown in FIGS. 2-4, these data identify Kvβ2 as a functional regulatory constituent of Kv1 channels that imparts stimulus-dependent redox control of vascular tone.

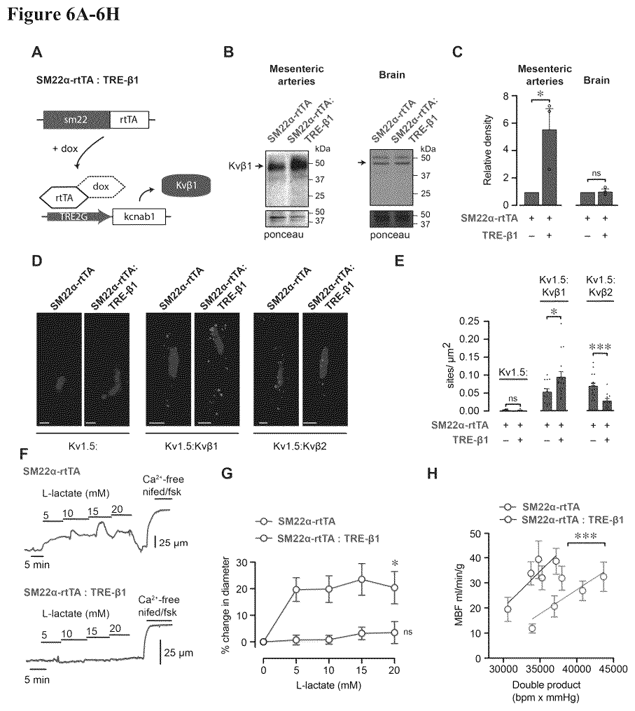

Increasing the Kvβ1.1: Kvβ2 ratio suppresses redox-dependent vasodilation and MBF. Native Kv1 channels are comprised of pore-forming subunits associated with more than one Kvβ subtype. This combinatorial variability may contribute to the diversity and cell-specific adaptability of channel function to a wide range of physiological and pathological stimuli. In coronary arterial myocytes, both Kvβ1.1 and Kvβ2 proteins are present in native Kv1 auxiliary subunit complexes; however, our data suggest that these proteins may have divergent roles in the regulation of arterial diameter and myocardial perfusion. That is, in contrast to our observations made in Kvβ2^{-/-} mice, deletion of Kvβ1.1 did not impede MBF. Structural comparison of the two subunits shows a clear difference in the N-termini of Kvβ 1 and Kvβ2 subunits. The N-termini of Kvβ1 proteins form a ball-and-chain-like inactivation domain, a feature that is lacking in Kvβ2. Thus, we hypothesized that the association of Kvβ1.1 with Kv1 channels may serve to counter the regulatory function imparted by Kvβ2. A testable prediction based on this hypothesis is that increasing the ratio of Kvβ1.1:Kvβ2 subunits in arterial myocytes would recapitulate the effects of Kvβ2 deletion. To examine this possibility, we generated transgenic mice with conditional doxycycline-inducible overexpression of Kvβ1.1 in smooth muscle cells (FIG. 6A, see Methods). Briefly, this model consists of transgenic mice with a reverse tetracycline trans-activator driven by the SM22a promoter (SM22α-rtTA) crossed to transgenic mice with Kcnab1 downstream of the tetracycline responsive element (TRE-Kvβ1) to yield double transgenic (SM22α-rtTA:TRE-Kvβ1) and single transgenic littermate control (SM22α-rtTA) mice. Western blot revealed elevated Kvβ1 protein abundance in arteries of SM22α-rtTA:TRE-Kvβ1 mice after doxycycline treatment, compared with arteries from doxycycline-treated SM22α-rtTA mice (FIG. 6B,C). Consistent with a lack of doxycycline effects on Kvβ1 protein in peripheral tissues, no statistically significant differences were observed in Kvβ1-associated band intensities in brain lysates of SM22α-rtTA:TRE-Kvβ1 versus SM22α-rtTA mice.

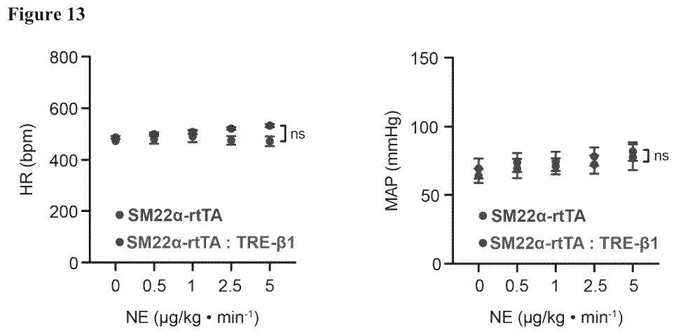

We next measured the relative levels of Kv1α:Kvβ protein interactions in coronary arterial myocytes via PLA. We observed PLA-associated fluorescent punctae in coronary arterial myocytes from SM22α-rtTA that were either co-labelled with Kv1.5 and Kvβ1, or with Kv1.5 and Kvβ2. Consistent with results of Western blot experiments described above, we observed a significant increase in Kv1.5:Kvβ1-associated PLA signal in coronary arterial myocytes from SM22α-rtTA:TRE-Kvβ1 when compared with myocytes from SM22α-rtTA mice (FIG. 6D, E). Notably, Kv1.5-Kvβ2-associated PLA signal was reduced in myocytes from SM22α-rtTA:TRE-Kvβ1 when compared with myocytes from SM22α-rtTA mice, suggesting that double transgenic mice express vascular Kv1 complexes with increased ratios of Kvβ1.1:Kvβ2 subunits. Functionally, enhanced Kvβ1.1:Kvβ2 subunit composition in arterial myocytes from SM22α-rtTA:TRE-Kvβ1 was associated with significantly blunted vasodilation of isolated mesenteric arteries in response to extracellular L-lactate when compared with arteries from single transgenic control mice (FIG. 6F, G). Indeed, these observations in SM22α-rtTA:TRE-Kvβ1 arteries were similar to those made in coronary and mesenteric arteries from Kvβ2^{-/-} mice, as well as arteries from WT mice pre-treated with the Kv1-selective inhibitor psora-4 (see FIGS. 4F-4I and 5C-5E). In vivo evaluation of the relationship between MBF and cardiac workload revealed significantly suppressed MBF in SM22α-rtTA:TRE-Kvβ1 mice when compared with SM22α-rtTA mice (FIG. 611). No differences in heart rate or MAP were observed between groups of mice (FIG. 13). Together, these results indicate that Kvβ1.1 in arterial myocytes functions as an inhibitory regulator of vasodilation, and that the control of MBF is balanced on the juxtaposing functional influences of Kvβ1.1 and Kvβ2 proteins.

### Discussion

In this study we identify vascular Kvβ proteins as key regulators of myocardial blood flow. Our findings suggest that the auxiliary Kvβ subunits impart oxygen sensitivity to Kv1 channel function, enabling them to trigger vasodilation in response to an increase in oxygen demand. A functional role of Kvβ proteins in imparting oxygen-sensitivity to Kv1 channels and thereby regulating vasodilation is supported by the following key findings: 1) Kvβ2^{-/-} mice exhibit acute cardiac failure during administration of norepinephrine; 2) MBF is significantly suppressed across the physiological range of cardiac workload in Kvβ2^{-/-} mice, yet is moderately enhanced in Kvβ1.1^{-/-} mice; 3) vasodilation of isolated coronary arteries in response to hypoxia and elevation of extracellular L-lactate is strongly attenuated by loss of Kvβ2; 4) whereas ablation of Kvβ proteins does not impact vasoconstriction of resistance caliber mesenteric arteries, vasodilation of these vessels in response to L-lactate is abolished by ablation of Kvβ2, comparable to effects of Kvβ2 deletion in coronary arteries; and 5) increasing the Kvβ1.1:Kvβ2 ratio in smooth muscle impairs L-lactate-induced vasodilation and suppresses MBF, similar to observations made in Kvβ2^{-/-} arteries and mice. Collectively these results support the concept that Kvβ1.1 and Kvβ2 cooperatively control vascular function and regulate MBF upon changes in metabolic demand.

Kv1 channels belong to one of several Kv subfamilies that regulate membrane potential and [Ca²⁺]ᵢ in arterial myocytes to control vessel diameter and blood flow.⁴¹ Pharmacological blockade of Kv1 channels reduces whole-cell outward I_{K} by ≥50%,⁴² whereas increased steady-state I_{Kv} results in membrane hyperpolarization and reduced Ca′ influx via voltage-gated Ca²⁺ channels. The resultant reduction in cytosolic [Ca²⁺]ᵢ leading to myocyte relaxation, and vasodilation increases local tissue perfusion. Considering the relatively high resting input resistance (1-10 GΩ) of arterial smooth muscle cells, the opening or closure of few K⁺ channels can generate substantial changes in membrane potential and vascular tone. Consequently, the functional expression of native Kv channels of arterial myocytes is dynamically controlled by multiple molecular processes, which include post-transcriptional regulation (e.g., phosphorylation, glycosylation), subcellular trafficking and recycling, redox modifications, as well as association with accessory subunits and regulatory proteins.^{21, 31, 46}-48 Adding to this complexity, our observation that deletion of Kvβ2 disrupts Kv1-dependent vasodilation is consistent with a functional role of this subunit in regulating the vasodilatory response to metabolic stress.

Kv channels in excitable cells assemble as either homomeric or heteromeric structures with varied α₄β₄ configurations of pore-forming and auxiliary subunits.⁴⁹⁻⁵² This ‘mix-and-match’ capability of Kv channels contributes to the wide heterogeneity of K⁺ currents that enables diverse physiological roles across different cell types. In our previous work we found that Kv1 channels in murine coronary arterial myocytes interact with Kvβ1.1/Kvβ2 heteromers,²⁰ and our present findings suggest a divergent functional regulation of vascular tone and blood flow by these proteins. These divergent roles are revealed by the observation that even though Kvβ2 ablation suppressed vasodilatory function and MBF, the loss of Kvβ1.1 had little impact on arterial diameter ex vivo, but elevated MBF in vivo. These findings suggest that Kvβ1 and β2 have somewhat divergent and potentially antagonist roles, which may relate to differences in their structures. The Kvβ1 has a ball-and-chain inactivation domain at the N-terminus, a feature that is lacking in Kvβ2. Potentially as a result of these differences, individual subunits have differential effects on the gating of non- and slowly-inactivating Kv1α channels. Specifically, Kvβ1 induces N-type inactivation in non-inactivating Kv1α proteins whereas Kvβ2 increases current amplitude and shifts the voltage-dependence of activation towards more hyperpolarized potentials, with little impact on channel inactivation. These effects are consistent with a greater steady-state activity of non-inactivating Kv1α channels (e.g., Kv1.5) when assembled with Kvβ2, as compared with those predominantly consisting of Kvβ1 proteins.

How the net competing influences of multiple Kvβ subtypes impact the function of native Kv1 channels remains to be resolved; however, it has been reported that within the same auxiliary complex, the N-terminal inactivation function of Kvβ1 is inhibited by Kvβ2 subunits, an effect which may be due to competition between Kvβ subtypes for the intracellular domain of pore-forming Shaker subunits, or through modification of Kvβ1 function via β:β subunit interactions. We found that in arterial myocytes both Kvβ1.1 and Kvβ2 proteins are expressed in native Kv1 channels, and therefore, it is plausible that the greater abundance of Kvβ2 relative to Kvβ1.1 in Kv1 channels of coronary arterial myocytes underlies its functional dominance under physiological conditions. Consistent with this are the apparent differences in inactivation kinetics between slowly inactivating outward K⁺ currents measured in coronary arterial myocytes in comparison with rapidly inactivating (i.e., A-type) currents recorded in retinal arteriolar myocytes, which predominantly express Kv1.5+Kvβ1 proteins. Indeed, our current data obtained from novel double transgenic mice overexpressing Kvβ1.1 in smooth muscle suggest that increased abundance of Kvβ1 proteins effectively diminishes the vasodilatory function attributed to Kvβ2. Thus, based on these findings, we speculate that Kvβ1 and β2 play antagonistic roles and that Kv channel remodeling which results in functional upregulation of Kvβ1.1 or downregulation of Kvβ2 (i.e., elevated Kvβ1.1:Kvβ2 ratio) could impair vasodilation and limit tissue perfusion.

The Kvβ proteins were discovered as functional AKRs, a group of enzymes that catalyze the reduction of carbonyl compounds by NAD(P)H. In our previous work, we found that the binding of oxidized and reduced pyridine nucleotides to Kvβ proteins differentially modifies channel gating, thus, raising the possibility that the Kvβ subunits provide a molecular link between the metabolic state of a cell and Kv channel activity. Given the high affinity of Kvβ proteins for pyridine nucleotides,^{14, 62} it is plausible that rapid changes in intracellular redox potential of pyridine nucleotides in arterial myocytes may underlie Kv-mediated control of blood flow in the heart upon changes in metabolic demand. We recently reported that Kvβ2 subunits facilitate surface expression of Kv1 and Kv4 channels in cardiomyocytes and that they impart redox and metabolic sensitivity to cardiac Kv channels, thus coupling repolarization with intracellular pyridine nucleotide redox status; however, to the best of our knowledge, the current study is the first to suggest a fundamental role for these subunits in controlling resistance vascular tone and blood flow.

Although our data show that Kvβ proteins regulate the diameter of resistance arteries subsequent to the modulation of NAD(H) redox via elevation of L-lactate, the precise identity of the factors responsible for coupling between myocardial oxygen consumption and coronary arterial tone remain unclear. Several myocardium-derived ‘metabolites’ (e.g., local O₂/CO₂ tensions, reactive oxygen species such as H₂O₂, lactate, endothelial-derived factors such as arachidonic acid metabolites) 9 could conceivably alter intracellular pyridine nucleotide redox potential and further work is required to identify specific metabolic processes that link intracellular redox changes to Kv activity. The function of coronary Kv1 channels could also be affected by other long-term biochemical processes. For example, the Kvβ proteins could plausibly alter patterns of basal post-transcriptional regulatory pathways (e.g., PKC-mediated channel phosphorylation) or the surface density of functional channels. However, such differences would likely manifest as differences in myogenic tone development or differential responses to vasoconstrictor stimuli, which were not seen in our study, suggesting that the vasoregulatory effects of Kvβ may reflect more dynamic modifications of channel function.

In summary, we report a novel role for intracellular Kvβ subunits in the differential regulation of resistance artery diameter and control of myocardial blood flow. Our results indicate that proper coupling between coronary arterial diameter and myocardial oxygen consumption relies on the molecular composition of Kv1 accessory subunit complexes such that the functional expression of Kvβ2 is essential for Kv1-mediated vasodilation. Moreover, the current study suggests that perturbations in Kvβ function or expression profile (i.e., Kvβ1.1:Kvβ2) may underlie the dysregulation of blood flow in disease states characterized by impaired microvascular function and ischemia-related cardiac dysfunction.

Although the foregoing specification and examples fully disclose and enable the present invention, they are not intended to limit the scope of the invention, which is defined by the claims appended hereto.

All publications, patents and patent applications are incorporated herein by reference. While in the foregoing specification this invention has been described in relation to certain embodiments thereof, and many details have been set forth for purposes of illustration, it will be apparent to those skilled in the art that the invention is susceptible to additional embodiments and that certain of the details described herein may be varied considerably without departing from the basic principles of the invention.

The use of the terms “a” and “an” and “the” and similar referents in the context of describing the invention are to be construed to cover both the singular and the plural, unless otherwise indicated herein or clearly contradicted by context. The terms “comprising,” “having,” “including,” and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein, including the best mode known to the inventors for carrying out the invention. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

With respect to ranges of values, the invention encompasses each intervening value between the upper and lower limits of the range to at least a tenth of the lower limit's unit, unless the context clearly indicates otherwise. Further, the invention encompasses any other stated intervening values. Moreover, the invention also encompasses ranges excluding either or both of the upper and lower limits of the range, unless specifically excluded from the stated range.

Further, all numbers expressing quantities of ingredients, reaction conditions, % purity, and so forth, used in the specification and claims, are modified by the term “about,” unless otherwise indicated. Accordingly, the numerical parameters set forth in the specification and claims are approximations that may vary depending upon the desired properties of the present invention. At the very least, and not as an attempt to limit the application of the doctrine of equivalents to the scope of the claims, each numerical parameter should at least be construed in light of the number of reported significant digits, applying ordinary rounding techniques. Nonetheless, the numerical values set forth in the specific examples are reported as precisely as possible. Any numerical value, however, inherently contains certain errors from the standard deviation of its experimental measurement.

Unless defined otherwise, the meanings of all technical and scientific terms used herein are those commonly understood by one of skill in the art to which this invention belongs. One of skill in the art will also appreciate that any methods and materials similar or equivalent to those described herein can also be used to practice or test the invention. Further, all publications mentioned herein are incorporated by reference in their entireties.

What is claimed is:

1. A method of modulating myocardial blood flow (MBF) as compared to a control in a patient in need thereof, comprising administering an agent that interacts with a Kvβ protein.

2. The method of claim 1, wherein the Kvβ protein is a Kvβ1 protein.

3. The method of claim 2, wherein the agent inhibits the Kvβ1 protein

4. The method of claim 1, wherein the Kvβ protein is a Kvβ2 protein.

5. The method of claim 2, wherein the agent inhibits the Kvβ2 protein

6. A method of suppressing myocardial blood flow (MBF) as compared to a control in a patient in need thereof, comprising administering an agent that inhibits a Kvβ protein.

7. The method of claim 6, wherein the Kvβ protein is a Kvβ1 protein.

8. The method of claim 7, wherein the agent inhibits the Kvβ1 protein

9. The method of claim 6, wherein the Kvβ protein is a Kvβ2 protein.

10. The method of claim 9, wherein the agent inhibits the Kvβ2 protein

11. A method of impairing cardiac contractile performance or arterial blood pressure as compared to a control in a patient in need thereof, comprising administering an agent that inhibits a Kvβ2 protein.

12. A method of reducing cardiac workload or preserving cardiac function during stress as compared to a control in a patient in need thereof, comprising administering an agent that inhibits a Kvβ1 protein.

13. A method of reducing L-lactate-induced vasodilation and suppression as compared to a control comprising administering an agent that interacts with a Kvβ protein and induces enhancement of a Kvβ1:Kvβ2 ratio in Kv1 channels of arterial smooth muscle.
